(19) United States
(12) Patent Application Publication
(10) Pub. No.: US 2024/0346653 A1
(43) Pub. Date: Oct. 17, 2024

(54) INFORMATION PROCESSING METHOD, INFORMATION PROCESSING APPARATUS, AND PROGRAM

(71) Applicants: TERUMO KABUSHIKI KAISHA, Tokyo (JP); Rokken Inc., Osaka (JP)

(72) Inventors: Yasukazu SAKAMOTO, Ashigarakami-gun (JP); Katsuhiko SHIMIZU, Ashigarakami-gun Kanagawa (JP); Hiroyuki ISHIHARA, Ashigarakami-gun (JP); Shunsuke YOSHIZAWA, Ashigarakami-gun Kanagawa (JP); Thomas HENN, Osaka (JP); Clement JACQUET, Osaka (JP); Stephen TCHEN, Osaka (JP); Ryosuke SAGA, Osaka (JP)

(73) Assignees: TERUMO KABUSHIKI KAISHA, Tokyo (JP); Rokken Inc., Osaka (JP)

(21) Appl. No.: 18/754,817

(22) Filed: Jun. 26, 2024

Related U.S. Application Data
(63) Continuation of application No. PCT/JP2022/047881, filed on Dec. 26, 2022.

(30) Foreign Application Priority Data
Dec. 28, 2021 (JP) 2021-214757

(51) Int. Cl. G06T 7/00; G06T 5/20; G06T 7/12; G06T 7/13; G06T 15/00; G06V 10/46; G06V 10/764

(52) U.S. Cl. CPC G06T 7/0012; G06T 5/20; G06T 7/12; G06T 7/13; G06T 15/00; G06V 10/46; G06V 10/764; G06T 2207/30021; G06T 2207/30024

(57) ABSTRACT

An information processing method for assisting a user to be capable of smoothly ascertaining necessary information. An information processing method causes a computer to execute processing for acquiring classification data in which pixels constituting biomedical image data indicating an internal structure of a living body are classified into a plurality of regions including a living tissue region in which a luminal region exists, the luminal region, and an extraluminal region outside the living tissue region, and generating region contour data, in which a portion where a thickness from an inner surface of the living tissue region facing the luminal region exceeds a predetermined threshold is removed from the living tissue region, based on the classification data.

### CROSS-REFERENCES TO RELATED APPLICATIONS

This application is a continuation of International Application No. PCT/JP2022/047881 filed on Dec. 26, 2022, which claims priority to Japanese Application No. 2021-214757 filed on Dec. 28, 2021, the entire content of both of which is incorporated herein by reference.

### TECHNOLOGICAL FIELD

The present disclosure generally relates to an information processing method, an information processing apparatus, and a program.

### BACKGROUND DISCUSSION

A catheter system that acquires a tomographic image by inserting an image acquisition catheter into a hollow organ such as a blood vessel is used (International Patent Application Publication No. WO 2017/164071).

A user such as a doctor ascertains information about a hollow organ, such as a running shape of the hollow organ, a state of an inner wall of the hollow organ, and a thickness of the hollow organ wall, using a tomographic image acquired by the catheter system.

However, in the catheter system of International Patent Application Publication No. WO 2017/164071, the user may not be able to smoothly ascertain necessary information due to a change in an invasion depth of the tomographic image depending on the state of the hollow organ.

### SUMMARY

In one aspect, an information processing method is disclosed that assists a user to be capable of smoothly ascertaining necessary information.

An information processing method causes a computer to execute processing, the processing including acquiring classification data in which pixels constituting biomedical image data indicating an internal structure of a living body are classified into a plurality of regions including a living tissue region in which a luminal region exists, the luminal region, and an extraluminal region outside the living tissue region, and generating region contour data in which a portion where a thickness from an inner surface of the living tissue region facing the luminal region exceeds a predetermined threshold is removed from the living tissue region, based on the classification data.

From one aspect, an information processing method for assisting a user to be capable of smoothly ascertaining necessary information can be provided.

### BRIEF DESCRIPTION OF THE DRAWINGS

FIG. 1 is an explanatory diagram describing an outline of a process for processing a tomographic image.

FIG. 2 is an explanatory diagram describing a configuration of an information processing apparatus.

FIG. 3 is an explanatory diagram describing a record layout of a tomographic image database (DB).

FIG. 4 is an explanatory diagram describing a classification model.

FIG. 5 is a flowchart describing a flow of processing of a program.

FIG. 6 is a screen example.

FIG. 7 is a screen example.

FIG. 8 is an explanatory diagram describing a configuration of a catheter system.

FIG. 9 is a flowchart describing a flow of processing of a program according to a second embodiment.

FIG. 10 is an explanatory diagram describing an outline of a process for processing a tomographic image according to a third embodiment.

FIG. 11 is a flowchart describing a flow of processing of a program according to the third embodiment.

FIG. 12 is an explanatory diagram describing a thickness of a living tissue region.

FIG. 13 is a flowchart describing a flow of processing of a program according to a fourth embodiment.

FIG. 14 is a screen example according to the fourth embodiment.

FIG. 15 is an explanatory diagram describing a thickness of a living tissue region in a modification.

FIG. 16 is an explanatory diagram describing classification data according to a fifth embodiment.

FIG. 17 is an explanatory diagram describing classification data according to the fifth embodiment.

FIG. 18 is a flowchart describing a flow of processing of a program according to the fifth embodiment.

FIG. 19 is a screen example according to the fifth embodiment.

FIG. 20 is an explanatory diagram describing a second classification model.

FIG. 21 is a flowchart describing a flow of processing of a program according to a modification.

FIG. 22 is an explanatory diagram describing an outline of a process for processing a tomographic image according to a sixth embodiment.

FIG. 23 is a flowchart describing a flow of processing of a program according to the sixth embodiment.

FIG. 24 is a flowchart describing a flow of processing of a program according to a modification.

FIG. 25 is an explanatory diagram describing a configuration of an information processing apparatus according to a seventh embodiment.

FIG. 26 is a functional block diagram of an information processing apparatus according to an eighth embodiment.

### DETAILED DESCRIPTION

Set forth below with reference to the accompanying drawings is a detailed description of embodiments of an information processing method, an information processing apparatus, and a program.

### First Embodiment

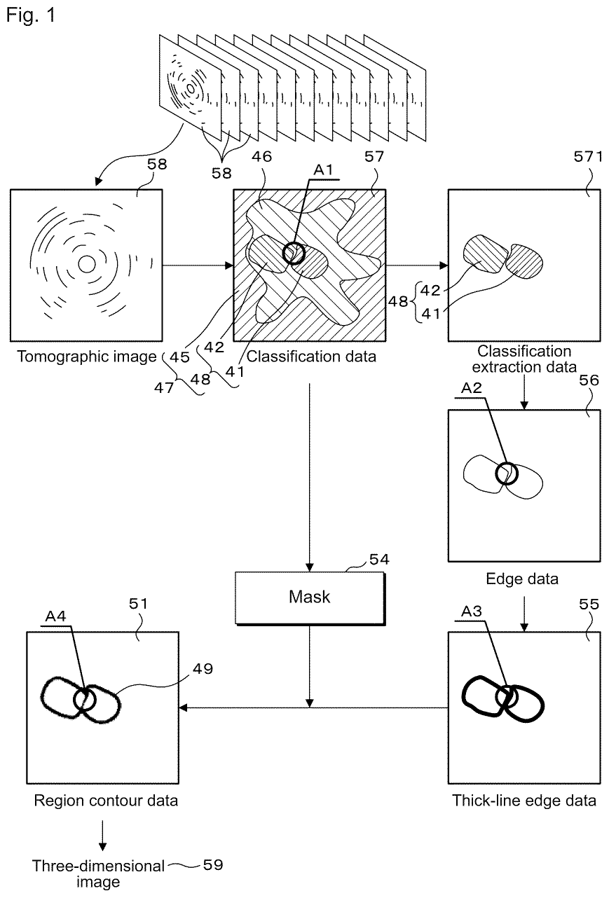

FIG. 1 is an explanatory diagram describing an outline of a process for processing a tomographic image 58. The tomographic image 58 is an example of a biomedical image generated based on biomedical image data indicating an internal structure of a living body.

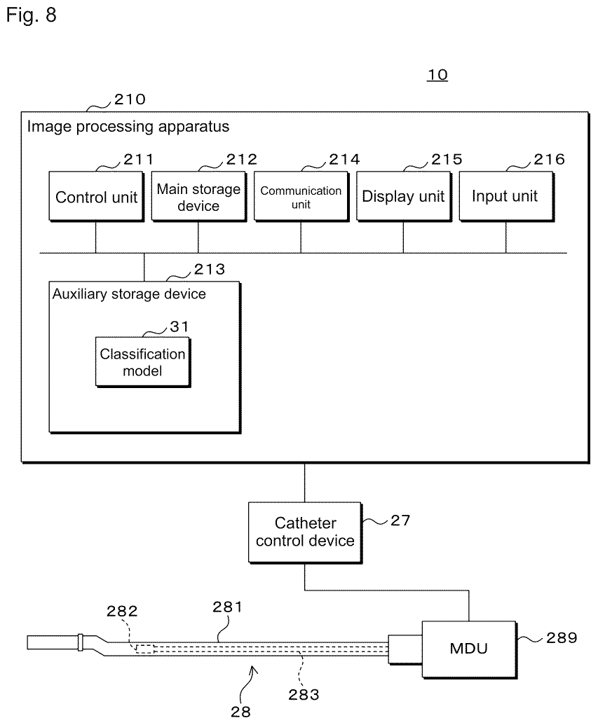

In the present embodiment, a described example is a case of using a plurality of tomographic images 58 generated in time series using a radial scanning type image acquisition catheter 28 (see FIG. 8). An example of the image acquisition catheter 28 will be described later. In the following description, the tomographic images 58 generated by one three-dimensional scanning may be referred to as one set of tomographic images 58. The data constituting the set of tomographic images 58 is an example of three-dimensional biomedical image data.

Note that, in FIG. 1, a so-called XY-format tomographic image 58 structured in accordance with an actual shape is illustrated as an example. The tomographic images 58 may be images of a so-called radiographic test (RT)-format in which scanning lines are arranged in parallel in order of scanning angles. During the processing described with reference to FIG. 1, conversion between the RT format and the XY format may be performed. Since the method of conversion between the RT format and the XY format are known, description of the method of conversion between the RT formal and XY format is omitted.

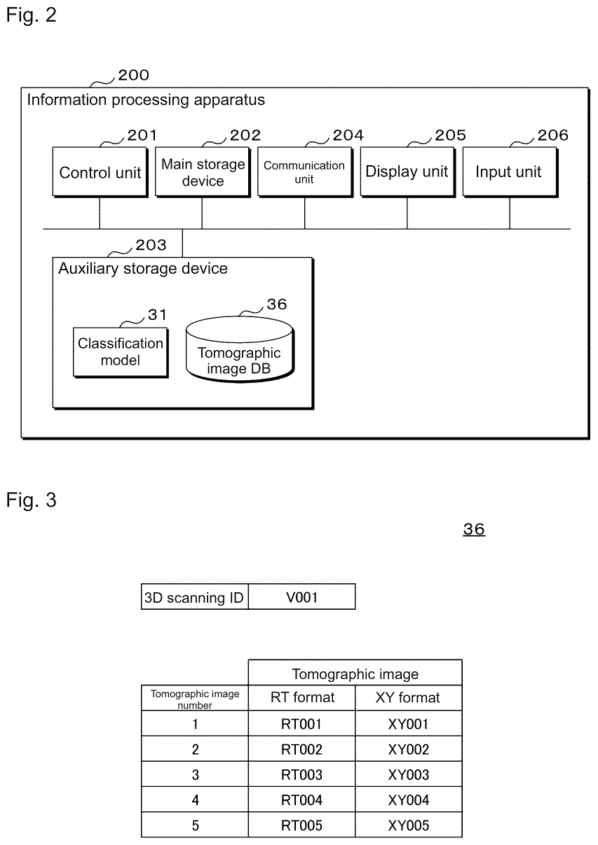

A control unit 201 (see FIG. 2) generates classification data 57 based on each tomographic image 58. The classification data 57 is data obtained by classifying pixels constituting the tomographic image 58 into a first luminal region 41, a second luminal region 42, an extraluminal region 45, and a living tissue region 46. One piece of classification data 57 is an example of two-dimensional classification data.

In the classification data 57, a label indicating any one of the first luminal region 41, the second luminal region 42, the living tissue region 46, and the extraluminal region 45 is given to each pixel constituting the tomographic image 58. A classification image, not illustrated, can be generated based on the classification data 57. The classification image can be, for example, an image in which pixels corresponding to the label of the first luminal region 41 are set to a first color, pixels corresponding to the label of the second luminal region 42 are set to a second color, pixels corresponding to the label of the living tissue region 46 are set to a third color, and pixels corresponding to the label of the extraluminal region 45 are set to a fourth color in the tomographic image 58.

FIG. 1 schematically illustrates the classification data 57 using the classification image. A portion of the first color corresponding to the first luminal region 41 is indicated by thin diagonally left-down hatching. A portion of the second color corresponding to the second luminal region 42 is indicated by thin diagonally right-down hatching. A portion of the third color corresponding to the living tissue region 46 is indicated by diagonally right-down hatching. A portion of the fourth color corresponding to the extraluminal region 45 is indicated by diagonally left-down hatching. “A1” indicates a portion where the living tissue region 46 is relatively thin or narrow in thickness in the classification data 57.

The first luminal region 41, the second luminal region 42, and the extraluminal region 45 are examples of a non-living tissue region 47. The first luminal region 41 and second luminal region 42 are examples of the luminal region 48 surrounded by living tissue region 46. Therefore, the boundary line between the first luminal region 41 and the living tissue region 46, and the boundary line between the second luminal region 42 and the living tissue region 46 indicate an inner surface of the living tissue region 46.

The first luminal region 41 is a lumen into which the image acquisition catheter 28 is inserted. The second luminal region 42 is a lumen into which the image acquisition catheter 28 is not inserted. The extraluminal region 45 is a region of the non-living tissue region 47 that is not surrounded by the living tissue region 46, that is, a region outside the living tissue region 46.

The control unit 201 generates classification extraction data 571 in which the pixels classified into the first luminal region 41 and the pixels classified into the second luminal region 42 are extracted from the classification data 57. In the classification extraction data 571, the region classified as the living tissue region 46 and the region classified as the extraluminal region 45 are not distinguished.

The control unit 201 can apply a known edge extraction filter to a classification extraction image generated based on the classification extraction data 571 to generate the edge data 56 in which the boundary line of the first color and the boundary line of the second color are extracted. The edge extraction filter can be, for example, a differential filter such as a Sobel filter or a Prewitt filter. Since the edge extraction filter is generally used in image processing, a detailed description of the edge extraction filter will be omitted.

The edge data 56 can be, for example, an image in which a thin black line corresponding to a boundary line is drawn on a white background. A portion corresponding to “A1” in the classification data 57 is indicated by “A2”. Two boundary lines are extracted close to each other.

The control unit 201 can generate, based on the edge data 56, thick-line edge data 55 in which the boundary line is thickened within a predetermined range to be a thick line. The thick-line edge data 55 can be, for example, an image in which a thick black line corresponding to a boundary line is drawn on a white background.

The thick-line edge data 55 can be generated by applying a known expansion filter to the edge data 56. Since the expansion filter is generally used in image processing, a detailed description of the expansion filter will be omitted. The portion corresponding to “A1” in the classification data 57 is indicated by “A3”. In the edge data 56, two adjacent boundary lines are fused to form one thick line.

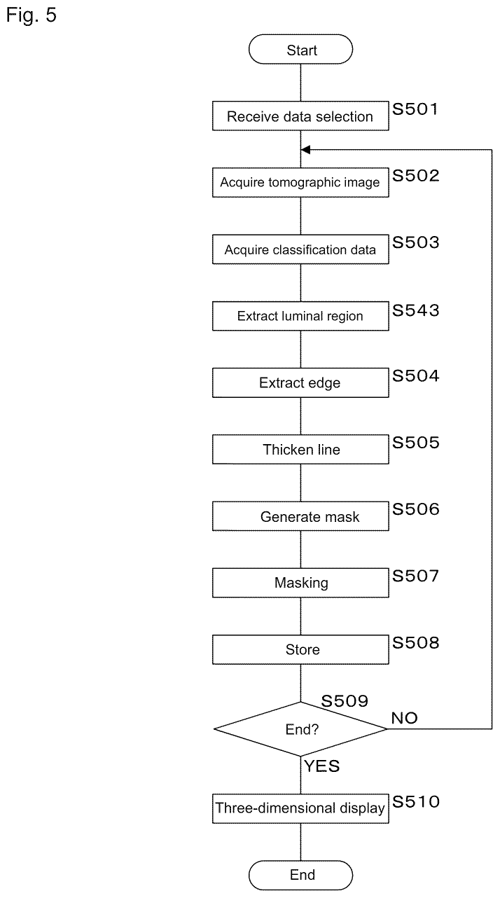

The control unit 201 generates a mask 54 corresponding to a pixel group classified into the living tissue region 46, based on the tomographic image 58 and the classification data 57. The mask 54 is a mask in which pixels corresponding to the label of the living tissue region 46 in the tomographic image 58 are set to “transparent” and pixels corresponding to the label of the non-living tissue region 47 are set to “opaque”. A specific example of the mask 54 will be described later in the description about steps S506 and S507 of the flowchart illustrated in FIG. 5.

The control unit 201 generates region contour data 51 by applying the mask 54 to the thick-line edge data 55 and extracting only the “transparent” portion of the thickened boundary line. The region contour data 51 can be an image in which, in the living tissue region 46, pixels of a portion having a distance from the inner surface being equal to or smaller than a predetermined threshold are black, and the other portion is white. The white portion can include both a portion of the living tissue region 46 where the distance from the inner surface exceeds the predetermined threshold and the non-living tissue region 47. The predetermined threshold corresponds to a predetermined thickness at a time of generating the thick-line edge data 55 based on the edge data 56.

In the following description, a black portion of the region contour data 51 may be referred to as a region contour area 49. The region contour data 51 generated based on one tomographic image 58 is an example of two-dimensional region contour data.

The portion in the region contour data 51 corresponding to “A1” in the classification data 57 is indicated by “A4”. The shape of the portion where the first luminal region 41 and the second luminal region 42 are close to each other and the living tissue region 46 is thinned is extracted clearly in the region contour area 49.

The control unit 201 generates three-dimensional image 59 based on the region contour data 51 generated based on each tomographic image 58. A user can smoothly ascertain the structure of the living tissue such as a thinned portion of the living tissue region 46 by observing the three-dimensional image 59 with the image being appropriately cut or rotated. Note that an example of the three-dimensional image 59 will be described later.

The control unit 201 may process only one tomographic image 58 and may not generate the three-dimensional image 59. In such a case, the control unit 201 outputs or stores the region contour data 51 or data in the middle of generating the region contour data 51 to or in the control unit 201 or the network.

FIG. 2 is an explanatory diagram describing a configuration of an information processing apparatus 200. The information processing apparatus 200 can include the control unit 201, a main storage device 202, an auxiliary storage device 203, a communication unit 204, a display unit 205, an input unit 206, and a bus. The control unit 201 is an arithmetic control device that executes the program of the present embodiment. As the control unit 201, one or a plurality of central processing units (CPUs), graphics processing units (GPUs), multi-core CPUs, or the like is used. The control unit 201 is connected to each hardware unit constituting the information processing apparatus 200 via the bus.

The main storage device 202 is a storage device such as a static random-access memory (SRAM), a dynamic random-access memory (DRAM), or a flash memory. The main storage device 202 temporarily stores information necessary during processing performed by the control unit 201 and a program being executed by the control unit 201.

The auxiliary storage device 203 is a storage device such as an SRAM, a flash memory, a hard disk, or a magnetic tape. The auxiliary storage device 203 stores a classification model 31, a tomographic image database (DB) 36, a program to be executed by the control unit 201, and various data necessary for executing the program. The classification model 31 and the tomographic image DB 36 may be stored in an external mass storage connected to the information processing apparatus 200. The communication unit 204 is an interface that performs communication between the information processing apparatus 200 and the network.

The display unit 205 can be, for example, a liquid crystal display panel, or an organic electro-luminescence (EL) panel. The input unit 206 can be, for example, a keyboard or a mouse. The display unit 205 and the input unit 206 may be laminated to constitute a touch panel.

The information processing apparatus 200 is a general-purpose personal computer, a tablet, a large-scale computer, a virtual machine that operates on a large-scale computer, or a quantum computer. The information processing apparatus 200 may include a plurality of personal computers that performs distributed processing, or hardware such as a large-scale computer. The information processing apparatus 200 may be configured by a cloud computing system.

FIG. 3 is an explanatory diagram describing a record layout of the tomographic image DB 36. The tomographic image DB 36 is a database in which tomographic image data indicating the tomographic image 58 generated by three-dimensional scanning is recorded. The tomographic image DB 36 can include a 3D scanning ID field, a tomographic image number field, and a tomographic image field. The tomographic image field has an RT format field and an XY format field.

In the 3D scan ID field, a 3D scanning ID assigned for each three-dimensional scanning is recorded. In the tomographic image number field, a number indicating the order of the tomographic images 58 generated by single three-dimensional scanning is recorded. In the RT format field, the RT format tomographic image 58 is recorded. In the XY format field, the XY format tomographic image 58 is recorded.

Note that only the tomographic image 58 of the RT format is recorded in the tomographic image DB 36, and the control unit 201 may generate the tomographic image 58 of the XY format by coordinate transformation as necessary. Instead of the tomographic image DB 36, a database in which data related to the scanning line before generation (or creation) of the tomographic image 58 and the like are recorded may be used.

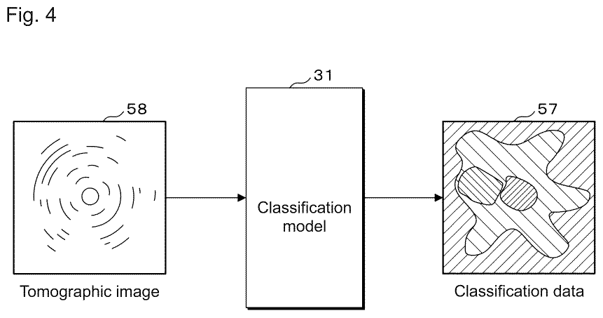

FIG. 4 is an explanatory diagram describing the classification model 31. The classification model 31 receives the tomographic image 58, classifies the pixels constituting the tomographic image 58 into the first luminal region 41, the second luminal region 42, the extraluminal region 45, and the living tissue region 46, and outputs data in which positions of the pixels are associated with the labels indicating the classification results.

The classification model 31 can be, for example, a trained model that performs semantic segmentation on the tomographic image 58. The classification model 31 can be a model generated by machine learning using training data, in which a large number of sets of the tomographic images 58 and correct answer data obtained by an expert such as a doctor differently coloring the first luminal region 41, the second luminal region 42, the extraluminal region 45, and the living tissue region 46 in the tomographic images 58, are recorded. Since the trained model for performing the semantic segmentation has been conventionally generated, the detailed description of the trained model for performing the semantic segmentation will be omitted.

Note that the classification model 31 may be a trained model trained to receive the tomographic image 58 of the RT format and output the classification data 57 of the RT format.

The classification data 57 in FIG. 4 is exemplified. In the classification model 31, the pixels constituting the tomographic image 58 may be classified into any region such as an instrument region corresponding to an instrument such as a guide wire used simultaneously with the image acquisition catheter 28, a calcification region, or a plaque region.

The classification model 31 may be a rule-based classifier. For example, in a case where the tomographic image 58 is an ultrasonic image, the pixels can be classified into respective regions based on luminance. For example, in a case where the tomographic image 58 is an X-ray computed tomography (CT) image, the classification model 31 can classify the pixels into respective regions based on luminance or CT values corresponding to the pixels.

FIG. 5 is a flowchart describing a flow of processing of a program. The control unit 201 receives selection of data to be three-dimensionally displayed from the user (step S501). For example, the user specifies a 3D scanning ID of the data desired to be displayed.

The control unit 201 searches the tomographic image DB 36 using the 3D scanning ID as a key, and acquires the tomographic image 58 having the smallest tomographic image number among the set of tomographic images 58 (step S502). The control unit 201 inputs the acquired tomographic image 58 to the classification model 31 and acquires the classification data 57 (step S503). The control unit 201 extracts pixels classified into the luminal region 48 from the classification data 57 (step S543). The classification extraction data 571 is generated in step S543.

The control unit 201 generates a classification extraction image based on the classification extraction data 571. The control unit 201 applies the edge extraction filter to the classification extraction image to generate the edge data 56 (step S504). The control unit 201 applies the expansion filter to the edge data 56 to generate thick-line edge data 55 obtained by thickening the line of the edge data 56 (step S505).

Note that, in the following description, a case will be described as an example in which the outer peripheral processing is performed so that the number of pixels does not change before and after applying filters in steps S504 and S505, and the number of pixels of the tomographic image 58, the number of pieces of the classification data, the number of the pixels of the edge data 56, and the number of the pixels of the thick-line edge data 55 match each other. Since the external peripheral processing is generally used in image processing, a detailed description of the external peripheral processing will be omitted.

The control unit 201 generates the mask 54 based on the classification data 57 (step S506). A specific example of the mask 54 will be described. The mask 54 is achieved by a mask matrix having the same number of rows and columns as the number of pixels in vertical and horizontal directions of the tomographic image 58. Matrix elements of the mask matrix are determined as follows based on the pixels of corresponding rows and columns in the tomographic image 58.

The control unit 201 acquires the label of the classification data 57 corresponding to the pixels constituting the tomographic image 58. In a case where the extracted label is the label of the living tissue region 46, the control unit 201 sets the matrix elements of the mask matrix corresponding to the pixels to “1”. In a case where the extracted label is the label of the non-living tissue region 47, the control unit 201 sets the matrix elements of the mask matrix corresponding to the pixels to “0”. The mask 54 is completed by performing the above processing on all the pixels constituting the tomographic image 58.

The control unit 201 performs masking processing for applying the mask 54 to the thick-line edge data 55 (step S507). A specific example of the masking processing will be described. The control unit 201 generates a thick-line edge matrix having the same number of elements as the number of pixels based on the thick-line edge data 55. In a case where the pixels of the thick-line edge data 55 are black pixels constituting the thick line, the corresponding matrix elements of the thick-line edge matrix are “1”. In a case where the pixels of the thick-line edge data 55 are white pixels not constituting the thick line, the corresponding matrix elements of the thick-line edge matrix are “0”.

The control unit 201 calculates a matrix in which a product of each element of the thick-line edge matrix and corresponding element of the mask matrix is an element. The calculated matrix is a region contour matrix corresponding to the region contour data 51. A relationship among the elements of the region contour matrix, the thick-line edge matrix, and the mask matrix is expressed by Formula (1).

$$Raj = Bij \times Mij \qquad ( 1 )$$

Element of i row and j column of the region contour matrix R is indicated by Rij.

An element of i rows and j columns of the thick-line edge matrix B are indicated by Bij.

An element of i row and j column of the mask matrix M are indicated by Mij.

The Formula (1) means that the region contour matrix R is calculated by the Hadamard product of the thick-line edge matrix B and the mask matrix M.

The control unit 201 generates, based on the Formula (1), a region contour matrix on a thick line in the thick-line edge data 55. In the region contour matrix, matrix elements corresponding to pixels classified into the living tissue region 46 are “1”, and matrix elements corresponding to the other pixels are “0” in the classification data 57. The pixels whose corresponding matrix elements are “1” are pixels included in the region contour area 49.

The region contour data 51 in which the mask 54 is applied to the thick-line edge data 55 is generated by replacing the region contour matrix calculated by the Formula (1) with an image in which “1” indicates black pixels and “0” indicates white pixels. The control unit 201 stores the generated region contour data 51 in the main storage device 202 or the auxiliary storage device 203 in association with the tomographic image number (step S508).

The control unit 201 determines whether processing on the set of tomographic images 58 has been completed (step S509). In a case where the processing is determined not to have been completed (NO in step S509), the control unit 201 returns to step S502 and acquires the next tomographic image 58. In a case where the processing is determined to have been completed (YES in step S509), the control unit 201 three-dimensionally displays the plurality of pieces of region contour data 51 stored in step S508 (step S510). The control unit 201 achieves the function of the output unit of the present embodiment by step S501. Thereafter, the control unit 201 ends the processing.

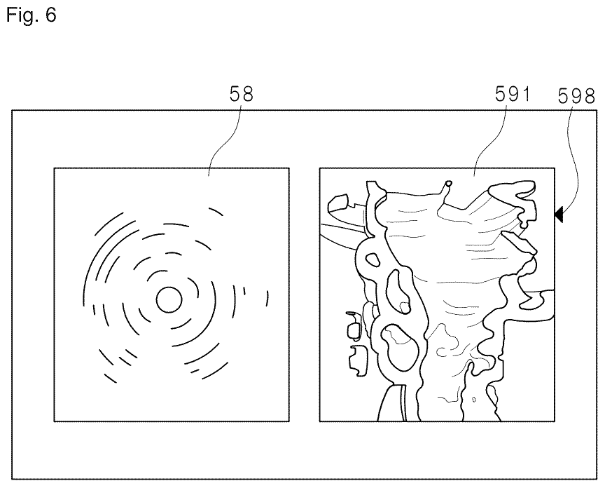

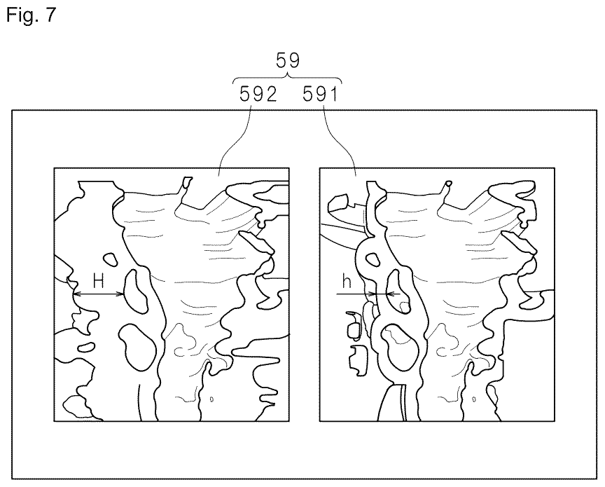

FIGS. 6 and 7 are screen examples. FIG. 6 illustrates the screen on which a first three-dimensional image 591 and the tomographic image 58 are displayed side by side. The first three-dimensional image 591 is the three-dimensional image 59 obtained by three-dimensionally structuring the plurality of pieces of region contour data 51. FIG. 6 illustrates the first three-dimensional image 591 in a state where the front side is removed by cutting along a plane parallel to a paper surface (i.e., a plane parallel to the three-dimensional image 591 as shown on the screen). The first three-dimensional image 591 expresses the three-dimensional shape of the region contour area 49.

The tomographic image 58 is one of the tomographic images 58 used for structuring the first three-dimensional image 591. A marker 598 displayed on the edge of the first three-dimensional image 591 indicates the position of the tomographic image 58. The user can appropriately change the tomographic image 58 to be displayed on the screen by dragging, for example, the marker 598.

FIG. 7 illustrates a screen on which two types of three-dimensional images 59, namely, the first three-dimensional image 591 and a second three-dimensional image 592 are displayed side by side. The second three-dimensional image 592 is the three-dimensional image 59 obtained by three-dimensionally structuring the plurality of pieces of classification data 57. The first three-dimensional image 591 and the second three-dimensional image 592 in FIG. 7 show the same orientation and cut surface.

In the second three-dimensional image 592, for example, a thick portion of the living tissue region 46 in a portion indicated by a thickness H is displayed as a relatively thick area. The left end of the arrow indicating the thickness H, that is, the position on the side far from the image acquisition catheter 28 is likely to be affected by artifacts due to attenuation of ultrasonic waves or the like inside a living tissue, and thus relatively high accuracy may be difficult to obtain.

The same portion as the thickness H is displayed with a predetermined thickness as indicated by a thickness h in the first three-dimensional image 591. By using the display format of the first three-dimensional image 591, the user can smoothly ascertain the structure of a hollow organ without being confused by the influence of artifacts or the like in the tomographic image 58.

The present embodiment can provide the information processing apparatus 200 that assists the user to be capable of smoothly ascertaining necessary information using the set of saved tomographic images 58.

As described above, the user can appropriately change the orientation and the cut surface of the three-dimensional image 59 using the known user interface. It is possible to provide the information processing apparatus 200 in which the shape and thickness of a relatively thin portion in the tomographic image 58 are clearly displayed in a case where the user displays a cross section including the portion indicated by “A4” in FIG. 1 in the three-dimensional image 59.

Note that the information processing apparatus 200 may not display the three-dimensional image 59. For example, in a case where the tomographic image 58 generated by using the image acquisition catheter 28 that is not for three-dimensional scanning is used, the information processing apparatus 200 may display the region contour data 51 instead of the three-dimensional image 59.

The tomographic image 58 is not limited to one generated by inserting the image acquisition catheter 28 into a hollow organ. For example, any medical image diagnostic apparatus, such as X-ray, X-ray computerized tomography (CT), magnetic resonance imaging (MRI), or external ultrasonic diagnostic apparatus, may be used for the generation of the tomographic image 58.

### Second Embodiment

The present embodiment relates to a catheter system 10 that acquires a tomographic image 58 in real time and performs three-dimensional display. Description of portions common to those in the first embodiment will be omitted.

FIG. 8 is an explanatory diagram describing a configuration of the catheter system 10. The catheter system 10 can include an image processing apparatus 210, a catheter control device 27, a motor driving unit (MDU) 289, and an image acquisition catheter 28. The image acquisition catheter 28 is connected to the image processing apparatus 210 via the MDU 289 and the catheter control device 27.

The image processing apparatus 210 can include a control unit 211, a main storage device 212, an auxiliary storage device 213, a communication unit 214, a display unit 215, an input unit 216, and a bus. The control unit 211 is an arithmetic control device that executes a program of the present embodiment. As the control unit 211, one or a plurality of CPUs, GPUs, multi-core CPUs, or the like can be used. The control unit 211 is connected to the respective hardware units constituting the image processing apparatus 210 via the bus.

The main storage device 212 is a storage device such as an SRAM, a DRAM, or a flash memory. The main storage device 212 temporarily stores information necessary during processing performed by the control unit 211 and a program being executed by the control unit 211.

The auxiliary storage device 213 is a storage device such as an SRAM, a flash memory, a hard disk, or a magnetic tape. The auxiliary storage device 213 stores a classification model 31, a program to be executed by the control unit 211, and various data necessary for executing the program. The communication unit 214 is an interface that performs communication between the image processing apparatus 210 and the network. The classification model 31 may be stored in an external mass storage device or the like connected to the image processing apparatus 210.

The display unit 215 can be, for example, a liquid crystal display panel, an organic EL panel, or the like. The input unit 216 can be, for example, a keyboard and a mouse. The display unit 215 and the input unit 216 may be laminated to constitute a touch panel. The display unit 215 may be a display device connected to the image processing apparatus 210.

The image processing apparatus 210 can be, for example, a general-purpose personal computer, a tablet, a large-scale computer, or a virtual machine that operates on a large-scale computer. The image processing apparatus 210 may include a plurality of personal computers that performs distributed processing, or hardware such as a large-scale computer. The image processing apparatus 210 may be configured by a cloud computing system. The image processing apparatus 210 and the catheter control device 27 may constitute integrated hardware.

The image acquisition catheter 28 includes a sheath 281, a shaft 283 inserted into the sheath 281, and a sensor 282 disposed at a tip of the shaft 283. The MDU 289 rotates, advances and retracts the shaft 283 and the sensor 282 inside the sheath 281.

The sensor 282 can be, for example, an ultrasonic transducer that transmits and receives ultrasonic waves, or a transmission and reception unit for optical coherence tomography (OCT) that emits near-infrared light and receives reflected light. The following will describe, as an example, a case where the image acquisition catheter 28 is an Intravascular Ultrasound (IVUS) catheter used when an ultrasonic tomographic image is captured from the inside of a circulatory organ.

The catheter control device 27 generates one tomographic image 58 at each rotation of the sensor 282. When the MDU 289 rotates the sensor 282 while pulling or pushing the sensor, the catheter control device 27 continuously generates the plurality of tomographic images 58 approximately perpendicular to the sheath 281. The control unit 211 sequentially acquires the tomographic images 58 from the catheter control device 27. In a manner described above, so-called three-dimensional scanning is performed.

The advancing and retracting operation of the sensor 282 includes both an operation for advancing and retracting the entire image acquisition catheter 28 and an operation for advancing and retracting the sensor 282 inside the sheath 281. The advancing and retracting operation may be automatically performed at a predetermined speed by the MDU 289 or may be manually performed by the user.

Note that the image acquisition catheter 28 is not limited to a mechanical scanning method for mechanically performing rotation, advancing, and retracting. For example, the image acquisition catheter 28 may be an electronic radial scanning type one using the sensor 282 in which a plurality of ultrasonic transducers is annularly disposed.

The image acquisition catheter 28 may implement the three-dimensional scanning by mechanically rotating or swinging the linear scanning type, convex scanning type, or sector scanning type sensor 282. Instead of the image acquisition catheter 28, for example, a transesophageal echocardiography (TEE) probe may be used.

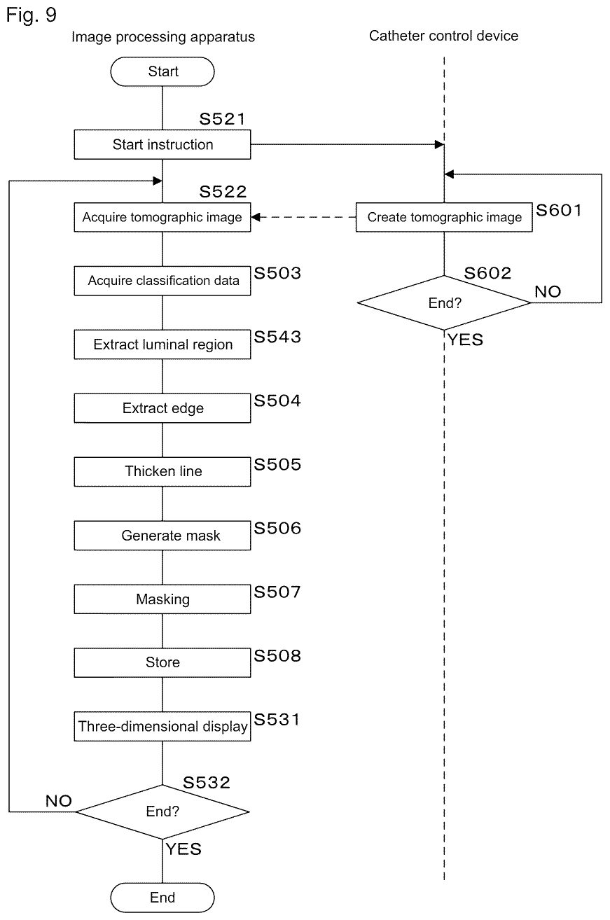

FIG. 9 is a flowchart describing a flow of the processing of the program according to the second embodiment. The control unit 211 receives an instruction to start scanning from the user (step S521). The control unit 211 instructs the catheter control device 27 to start scanning. The catheter control device 27 generates the tomographic image 58 (step S601).

The catheter control device 27 determines whether the three-dimensional scanning has been completed (step S602). In a case where the processing is determined not to have been completed (NO in step S602), the catheter control device 27 returns to step S601 and generates the next tomographic image 58. In a case where the processing is determined to have been completed (YES in step S602), the catheter control device 27 returns the sensor 282 to an initial position and shifts to a standby state of waiting for an instruction from the control unit 211.

The control unit 211 acquires the generated tomographic image 58 (step S522). Hereinafter, since the processing from step S503 to step S508 is the same as the processing of the program in the first embodiment described with reference to FIG. 5, the description of the processing from step S503 to step S508 will be omitted.

The control unit 211 three-dimensionally displays the plurality of pieces of region contour data 51 stored in step S508 (step S531). The control unit 211 determines whether the catheter control device 27 has completed the single three-dimensional scanning (step S532). In a case where the processing is determined not to have been completed (NO in step S532), the control unit 211 returns to step S522. In a case where the processing is determined to have been completed (YES in step S532), the control unit 211 ends the processing.

According to the present embodiment, the user can observe the three-dimensional image 59 in real time during the three-dimensional scanning.

Note that the control unit 211 may temporarily record the tomographic image 58 acquired in step S522 in the tomographic image DB 36 described with reference to FIG. 3, and execute the processing in and after step S503 while sequentially reading.

Note that the image acquisition catheter 28 is not limited to the three-dimensional scanning. The catheter system 10 may include the image acquisition catheter 28 dedicated to two-dimensional scanning, and the information processing apparatus 200 may display the region contour data 51 instead of the three-dimensional image 59.

### Third Embodiment

The present embodiment relates to an information processing apparatus 200 in which a user specifies a target region for which region contour data 51 is to be generated. Description of portions common to those in the first embodiment will be omitted.

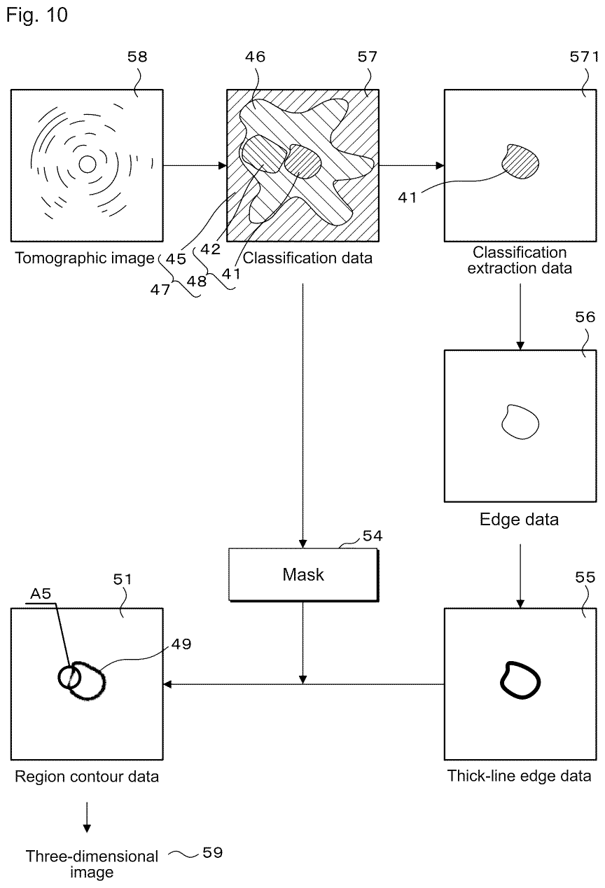

FIG. 10 is an explanatory diagram describing an outline of a process for processing a tomographic image 58 according to a third embodiment. A control unit 201 inputs the tomographic image 58 to a classification model 31 and acquires a classification data 57 that is output.

The control unit 201 receives selection of a region from the user. FIG. 10 illustrates an example of a case where the user selects a first luminal region 41. The control unit 201 generates classification extraction data 571 obtained by extracting pixels classified into the first luminal region 41 from the classification data 57. In the classification extraction data 571, a region classified as a living tissue region 46, a region classified as an extraluminal region 45, and a region classified as a second luminal region 42 are not distinguished.

The control unit 201 applies a known edge extraction filter to a classification extraction image generated based on the classification extraction data 571 to generate the edge data 56 in which the boundary line of the first luminal region 41 is extracted. The control unit 201 generates a thick-line edge data 55 based on the edge data 56.

The control unit 201 generates a mask 54 in which pixels corresponding to the label of the living tissue region 46 in the tomographic image 58 are set to “transparent” and pixels corresponding to the label of a non-living tissue region 47 are set to “opaque”.

The control unit 201 generates region contour data 51 by applying the mask 54 to the thick-line edge data 55 and extracting only the “transparent” portion of the thickened boundary line. The control unit 201 generates a three-dimensional image 59 based on the region contour data 51 generated based on each tomographic image 58. The user can observe the three-dimensional structure of a first luminal region 41 through the three-dimensional image 59.

The portion corresponding to “A4” in FIG. 1 is indicated by “A5” in FIG. 10. As in the first embodiment, it is clearly illustrated that the portion indicated by “A5” is relatively thin.

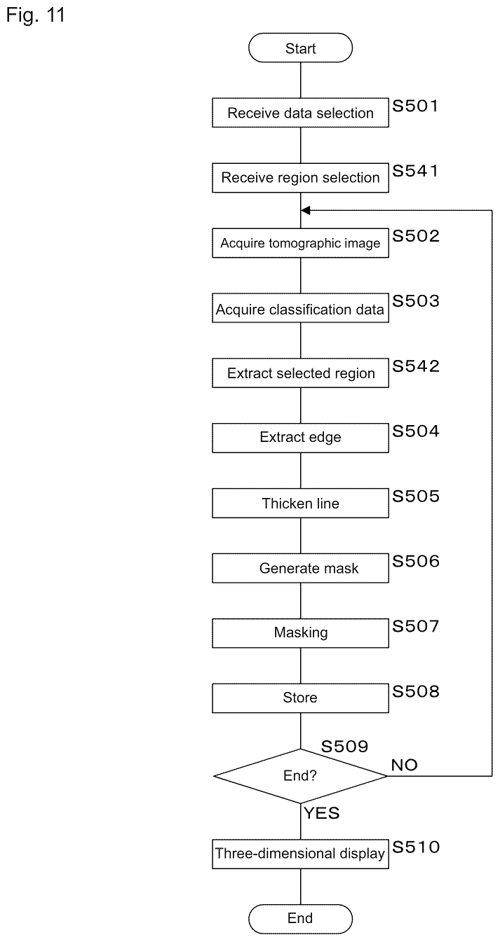

FIG. 11 is a flowchart describing a flow of the processing of a program according to the third embodiment. The control unit 201 receives selection of data to be three-dimensionally displayed from the user (step S501). For example, the user specifies a 3D scanning ID of the data desired to be displayed.

The control unit 201 receives selection of a region from the user (step S541). The user can select, for example, either the first luminal region 41 or the second luminal region 42, or both luminal region 48. In a case where the plurality of second luminal regions 42 is delineated, the user can select any one or the plurality of second luminal regions 42. FIG. 10 illustrates an example in which selection of the first luminal region 41 is received. In the following description, the luminal region 48 received for selection in step S541 is referred to as a selected region.

The control unit 201 searches the tomographic image DB 36 using the 3D scanning ID as a key and acquires the tomographic image 58 having the smallest tomographic image number among the set of tomographic images 58 (step S502). The control unit 201 inputs the acquired tomographic image 58 to the classification model 31 and acquires the classification data 57 (step S503).

The control unit 201 extracts, from the classification data 57, the selected region whose selection has been received in step S541 (step S542). Specifically, the control unit 201 generates classification data 57 in which the label related to the selected region whose selection has been received in step S541 is left, and the label related to a region other than the selected region is changed to a label indicating the “other” region.

The control unit 201 generates a classification extraction image based on the classification extraction data 571. The control unit 201 applies the edge extraction filter to the classification extraction image to generate the edge data 56 (step S504). Since subsequent processing is the same as the flow of the processing of the program described with reference to FIG. 5, the description of the flow of the subsequent processing will be omitted.

The present embodiment can provide the information processing apparatus 200 that assists the user to be capable of smoothly ascertaining necessary information with attention being paid to a specific region. As described about the portion indicated by “A5” in FIG. 10, the information processing apparatus 200 that clearly extracts a thin portion in the tomographic image 58 and performs three-dimensional display can be provided.

As in the second embodiment, the three-dimensional image 59 may be displayed in real time by the image processing apparatus 210.

### Fourth Embodiment

The present embodiment relates to an information processing apparatus 200 that displays a portion where a thickness of a living tissue region 46 exceeds a predetermined threshold in a mode different from the other portions. Description of the portions common to those in the first embodiment will be omitted.

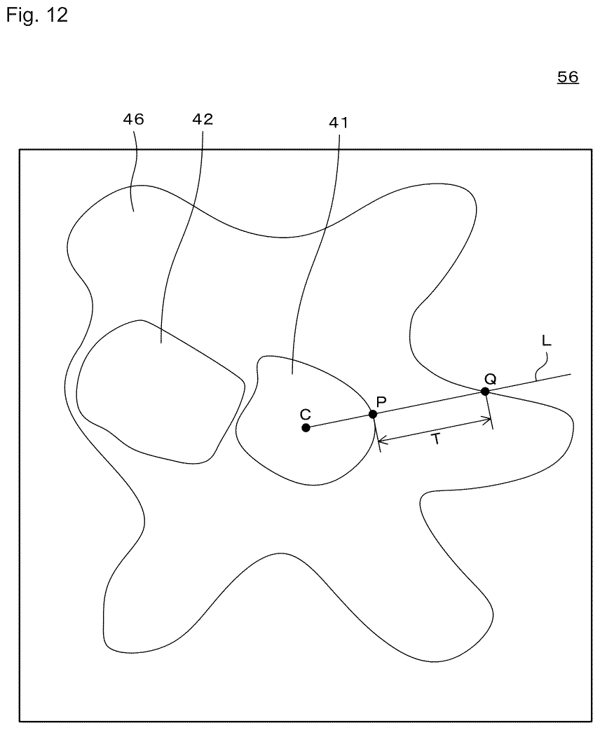

FIG. 12 is an explanatory diagram describing a thickness of the living tissue region 46. In FIG. 12, a point C indicates the center of edge data 56, that is, the rotation center of an image acquisition catheter 28. A point P is an intersection of a straight-line L passing through the point C and a boundary line inside the living tissue region 46. A point “Q” is an intersection of the straight-line L and a boundary line outside the living tissue region 46. The point P corresponds to the inner surface of the living tissue region 46. The point Q corresponds to the outer surface of the living tissue region 46.

A distance between P and Q is defined as a thickness T of the living tissue region 46 at the point P. In the following description, a portion having the thickness T exceeding a predetermined thickness is referred to as a thick portion. The predetermined thickness can be, for example, 1 centimeter.

The straight-line L corresponds to one scanning line in radial scanning. The information processing apparatus 200 calculates the thickness T for each scanning line with which a tomographic image 58 has been generated. The information processing apparatus 200 may calculate the thickness T for scanning lines at predetermined intervals such as every 10 scanning lines.

Note that the center of gravity of a first luminal region 41 may be used as the point C. By calculating the thicknesses T along lines radially defined through the center of gravity, even if the rotation center of the image acquisition catheter 28 is located near the inner surface of the first luminal region 41, the user can measure thickness T that closely matches their perception.

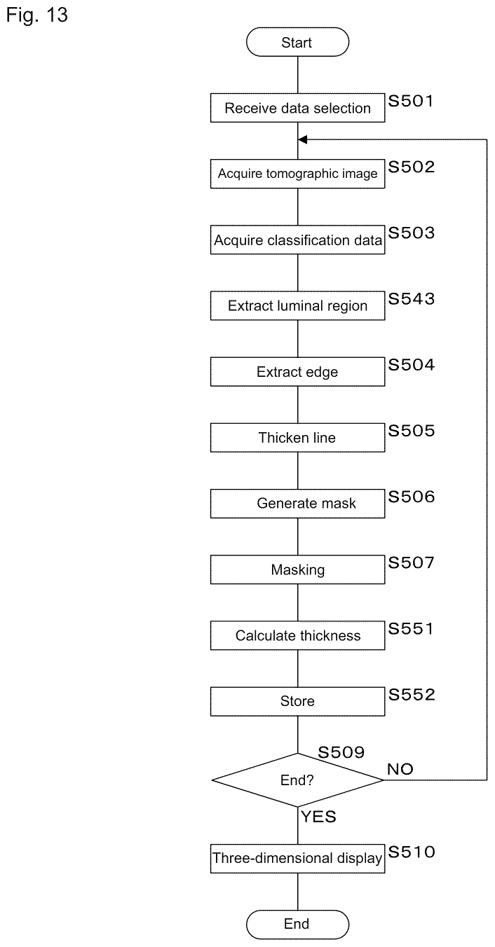

FIG. 13 is a flowchart describing a flow of the processing of a program according to the fourth embodiment. Since the processing up to step S507 is similar to the processing of the program in the first embodiment described with reference to FIG. 5, the description of the processing up to step S507 will be omitted.

The control unit 201 calculates the thickness T of the living tissue region 46 for each scanning line as described with reference to FIG. 12, based on the edge data 56 generated in step S504 (step S551). The control unit 201 stores a tomographic image number, region contour data 51, and the thickness T for each scanning line in association with each other in a main storage device 202 or an auxiliary storage device 203 (step S552).

The control unit 201 determines whether processing on one set of tomographic images 58 has been completed (step S509). In a case where the processing is determined not to have been completed (NO in step S509), the control unit 201 returns to step S502 and acquires the next tomographic image 58. In a case where the processing is determined to have been completed (YES in step S509), the control unit 201 three-dimensionally displays the plurality of pieces of region contour data 51 stored in step S508 (step S510). Thereafter, the control unit 201 ends the processing.

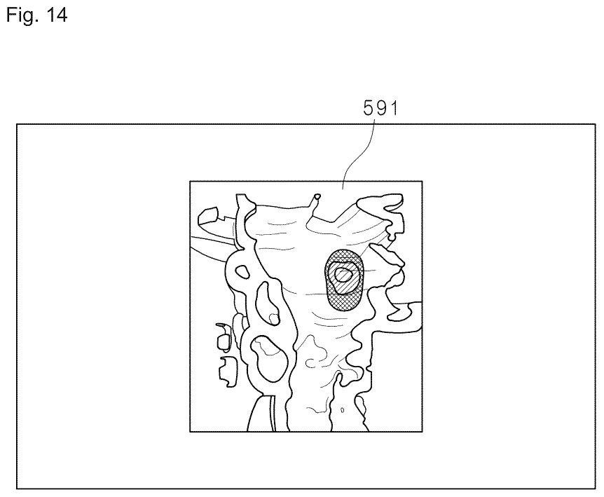

FIG. 14 is a screen example according to the fourth embodiment. The control unit 201 assigns display color data determined in accordance with the thickness T calculated in step S551 described with reference to FIG. 13 to pixels corresponding to the inner surface or the outer surface of a contour of a region contour area 49. Here, the inner surface of the region contour area 49 is identical to the inner surface of the living tissue region 46 and corresponds to the point P described using FIG. 12. The outer surface of the region contour area 49 corresponds to the intersection of the straight-line L and the boundary on the distal side of the region contour area 49. In FIG. 14, the difference in the display color data assigned to the inner surface of the living tissue region 46 is schematically illustrated by the types of hatching.

According to the present embodiment, a user can rather easily recognize the original thickness of the living tissue region 46 by using the color determined in advance in accordance with the thickness T.

### Modification

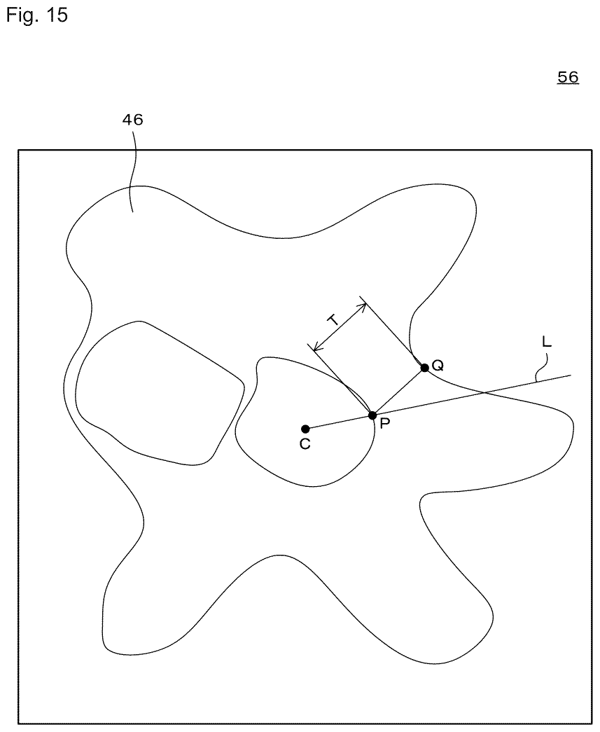

FIG. 15 is an explanatory diagram describing a thickness of the living tissue region 46 in a modification. In the present modification, a point closest to the point P on the boundary line outside the living tissue region 46 is defined as a point Q. Note that, after the three-dimensional image 59 is generated, the point three-dimensionally closest to the point P on the boundary surface outside of the living tissue region 46 may be defined as the point Q. The control unit 201 calculates the thickness T, which is a distance between the points P and Q.

### Fifth Embodiment

The present embodiment relates to a control unit 201 that generates three-dimensional region contour data by generating boundary surface data indicating a boundary surface between a luminal region 48 and a living tissue region 46 and a three-dimensional mask 54 after a three-dimensional image 59 is generated. Description of the portions common to those in the third embodiment will be omitted.

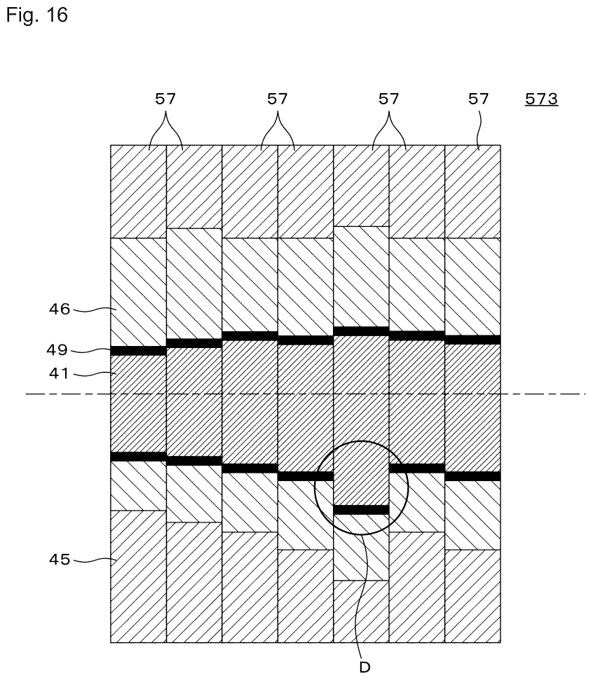

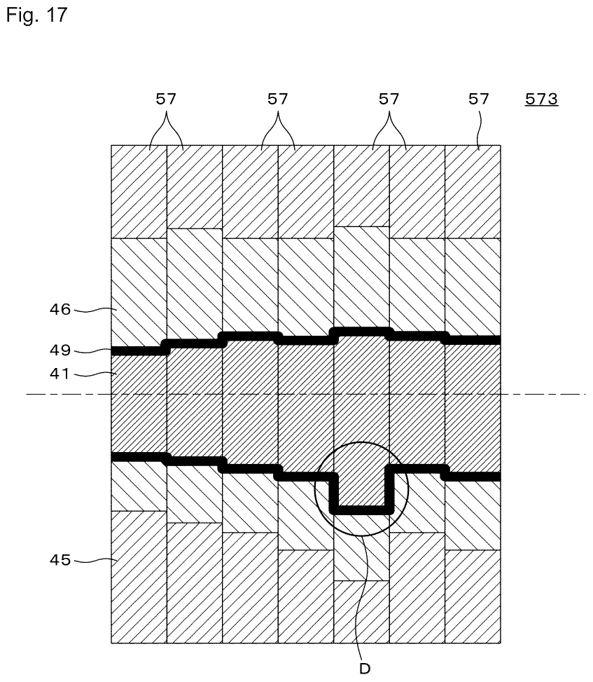

FIGS. 16 and 17 are explanatory diagrams describing classification data 57 according to the fifth embodiment. FIG. 16 schematically illustrates three-dimensional classification data 573 generated based on the plurality of classification data 57 generated with the method of the first embodiment.

The three-dimensional classification data 573 corresponds to a three-dimensional classification image configured by laminating classification images respectively corresponding to one set of tomographic images 58 with a thickness similar to the interval between the tomographic images 58. Note that when the three-dimensional classification data 573 is generated, the control unit 201 may perform interpolation in a thickness direction of the classification data 57 to smoothly connect the classification data 57 to each other.

FIG. 16 schematically illustrates the three-dimensional classification data 573 using a cross section obtained by cutting the three-dimensional classification image along a cross section passing through the central axis of the image acquisition catheter 28. A dot-and-dash line indicates the central axis of the image acquisition catheter 28. A black portion indicates a region contour area 49.

In the portion indicated by D in FIG. 16, a first luminal region 41 extends downward in FIG. 16 beyond the width of the region contour area 49. Therefore, the adjacent region contour areas 49 are not connected to each other, and when the region contour area 49 is three-dimensionally displayed, a through hole is drawn in an opened state.

However, in an actual hollow organ, an event such that a through hole is generated only in a portion corresponding to one of the tomographic images 58 is unlikely to occur. Such a through hole that does not actually exist becomes an obstacle when a doctor or the like observes the three-dimensional image 59 to quickly ascertain a three-dimensional structure of the hollow organ.

FIG. 17 schematically illustrates the region contour area 49 generated in the present embodiment. In the present embodiment, after generating classification extraction data 571 from the classification data 57, the control unit 201 structures a three-dimensional image of the luminal region 48. Thereafter, when edge extraction is performed on the three-dimensional image, a thin boundary surface that smoothly covers a portion corresponding to the through hole described with reference to FIG. 16 is extracted.

The control unit 201 generates a thick boundary surface in which a predetermined thickness is imparted to the extracted surface. The thick boundary surface is a range through which a sphere passes when the center of the sphere moves three-dimensionally along the boundary surface. The thickness of the thick boundary surface corresponds to the diameter of the sphere.

The control unit 201 generates a three-dimensional mask 54 based on the three-dimensional classification data 573. The control unit 201 applies the three-dimensional mask 54 to the thick boundary surface to generate three-dimensional region contour data indicating the three-dimensionally continuous region contour area 49. As described above, the control unit 201 can generate the region contour area 49 having a smooth shape without the through hole as illustrated in FIG. 17.

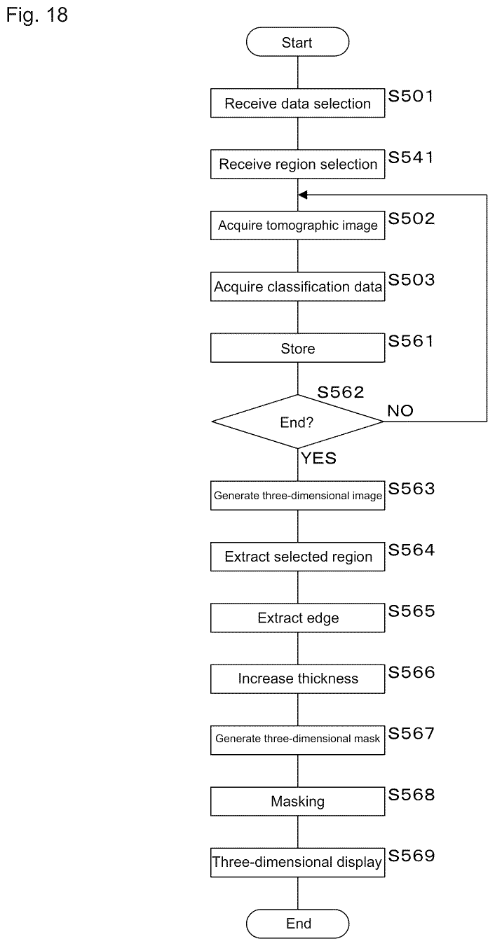

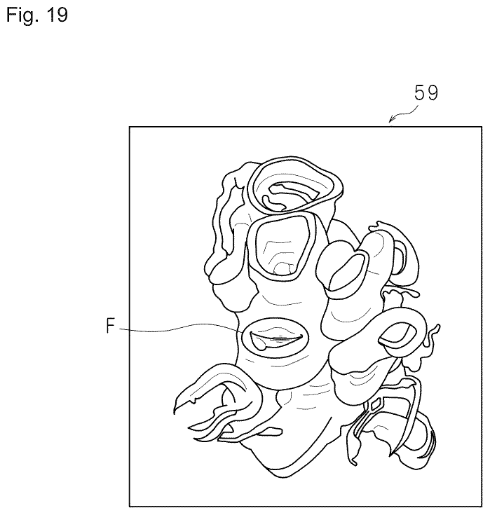

FIG. 18 is a flowchart describing a flow of the processing of a program according to the fifth embodiment. Since the flow of the processing of the program in steps S501 to S503 is similar to that in the third embodiment described with reference to FIG. 12, the description of the processing of the program in steps S501 to S503 will be omitted.

The control unit 201 stores the acquired classification data 57 in a main storage device 202 or an auxiliary storage device 203 in association with a tomographic image number (step S561). The control unit 201 determines whether processing on the set of tomographic images 58 has been completed (step S562). In a case where the processing is determined not to have been completed (NO in step S562), the control unit 201 returns to step S502 and acquires the next tomographic image 58.

In a case where the processing is determined to have been completed (YES in step S562), the control unit 201 generates three-dimensional classification data 573 based on the set of classification data 57 stored in step S561 (step S563). When the three-dimensional classification data 573 is generated, the control unit 201 may perform interpolation in a thickness direction of the tomographic image 58 to smoothly connect classification images corresponding to the respective tomographic images 58.

In the following description, a three-dimensional pixel constituting the three-dimensionally classification image is referred to as a “voxel”. A quadrangular prism, in which the dimension of one pixel in the tomographic image 58 is the dimension of a bottom surface and the distance between the adjacent tomographic images 58 is the height, includes a plurality of voxels. Each voxel has information regarding a color defined for each label of the classification data 57.

The control unit 201 extracts a three-dimensional selected region whose selection has been received in step S541 from the three-dimensional classification image (step S564). As described above, the selected region is a region selected by a user from the luminal region 48. In the following description, an image corresponding to the three-dimensional selected region may be referred to as a region three-dimensional image.

The control unit 201 generates boundary surface data, which is three-dimensional edge data 56 obtained by extracting a boundary surface of the region three-dimensional image, by applying a known three-dimensional edge extraction filter to the region three-dimensional image (step S565). The boundary surface data indicates a three-dimensional image with a thin membrane which is disposed in a three-dimensional space and indicates the boundary surface between the luminal region 48 and the living tissue region 46. Since the three-dimensional edge extraction filter is known, a detailed description of the three-dimensional edge extraction filter will be omitted.

The control unit 201 generates thick boundary surface data in which the boundary surface is thickened by giving a thickness within a predetermined range, based on the boundary surface data (step S566). The thick boundary surface data can be generated, for example, by applying a known three-dimensional expansion filter to the boundary surface data. The thick boundary surface data indicates a three-dimensional image with a thick membrane that indicates the boundary surface between the luminal region 48 and the living tissue region 46 and is disposed in a three-dimensional space. Since the three-dimensional expansion filter is known, a detailed description of the three-dimensional expansion filter will be omitted.

The control unit 201 generates the three-dimensional mask 54 based on the three-dimensional classification data 573 generated in step S563 (step S567). Specific examples will be described. The mask 54 is achieved by a mask matrix that is a three-dimensional matrix having the same number of matrix elements as the number of voxels in the vertical, horizontal, and height directions of the three-dimensional image 59. Each matrix element of the mask matrix is defined as follows based on a voxel at a corresponding position in the three-dimensional image 59.

The control unit 201 acquires the color of the voxels constituting the three-dimensional image 59. In a case where the acquired color is a color corresponding to the living tissue region 46, the control unit 201 sets the matrix elements of the mask matrix corresponding to the voxels to “1”. In a case where the acquired color is a color that does not correspond to the living tissue region 46, the control unit 201 sets the matrix elements of the mask matrix corresponding to the voxels to “0”. By performing the above processing on all the voxels constituting the three-dimensional image 59, the three-dimensional mask 54 is completed.

The control unit 201 performs masking processing for applying the three-dimensional mask 54 generated in step S567 to the thick boundary surface data generated in step S566 (step S568). By the masking processing, three-dimensional region contour data 51 is completed.

The control unit 201 displays completed three-dimensional region contour data 51 (step S569). In step S569, the three-dimensional shape of the region contour area 49 is displayed on a display unit 205. Thereafter, the control unit 201 ends the processing.

FIG. 19 is a screen example according to the fifth embodiment. A portion F in FIG. 19 corresponds to a portion D in FIG. 17. According to the present embodiment, the three-dimensional image 59 having no through hole can be displayed by performing the three-dimensional interpolation and masking processing.

### Modification

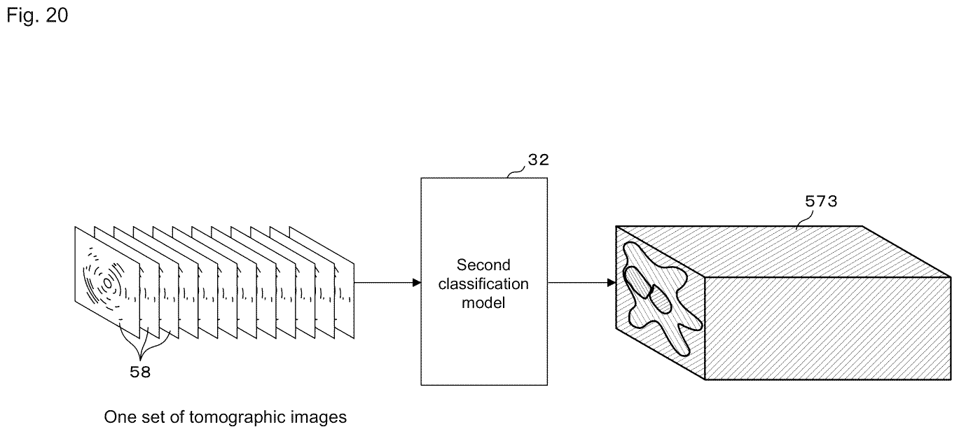

A modification using a second classification model 32 that receives an input of one set of tomographic images 58 and outputs the three-dimensional classification data 573 will be described. FIG. 20 is an explanatory diagram describing the second classification model 32. The second classification model 32 receives the set of tomographic images 58 and outputs the three-dimensional classification data 573.

The second classification model 32 can be, for example, a trained model that performs three-dimensional semantic segmentation on the set of tomographic images 58. The second classification model 32 is a model generated by machine learning using training data including a great number of sets of the set of tomographic images 58 and correct answer data, which is three-dimensionally structured after an expert such as a doctor differently colors the first luminal region 41, the second luminal region 42, the extraluminal region 45, and the living tissue region 46 in each tomographic image 58. The second classification model 32 may be a rule-based classifier.

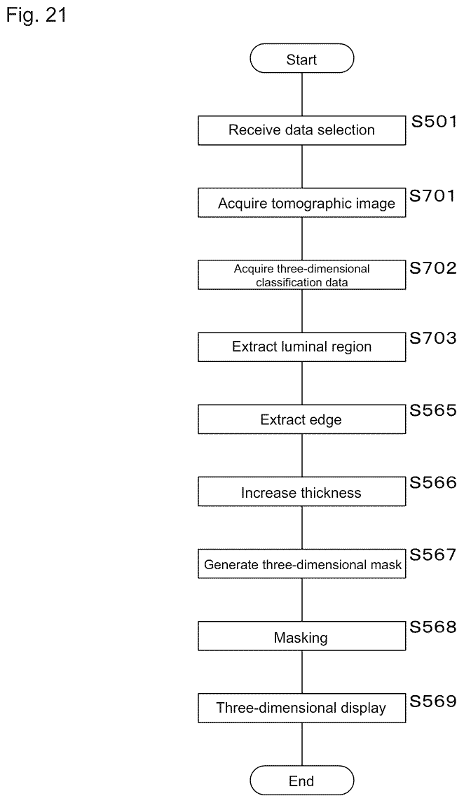

FIG. 21 is a flowchart describing the flow of processing of the program according to the modification. The control unit 201 receives selection of data to be three-dimensionally displayed from the user (step S501). The control unit 201 searches the tomographic image DB 36 using the 3D scanning ID as a key to acquire one set of tomographic images 58 (step S701).

The control unit 201 inputs the one set of tomographic image 58 to the second classification model 32 and acquires the three-dimensional classification data 573 (step S702). The control unit 201 extracts a region three-dimensional image corresponding to the three-dimensional shape of the living tissue region 46 from the three-dimensional classification data 573 (step S703).

The control unit 201 generates boundary surface data by applying a known three-dimensional edge extraction filter to the region three-dimensional image (step S565). Since subsequent processing is similar to the flow of the processing in the fifth embodiment described with reference to FIG. 18, the description of the flow of the processing in the fifth embodiment described with reference to FIG. 18 will be omitted.

According to the present modification, since the classification data 57 does not have to be acquired based on each tomographic image 58, the information processing apparatus 200 that performs three-dimensional display at high speed can be provided.

Note that the second classification model 32 may be a model that receives an input of a three-dimensionally structured image based on the set of tomographic images 58 and outputs the three-dimensional classification data 573. Three-dimensional classification data 573 can be quickly generated based on an image generated by a medical image diagnostic apparatus capable of generating a three-dimensional image without using the tomographic image 58.

### Sixth Embodiment

The present embodiment relates to a control unit 201 that generates a thick-line edge data 55 without via edge data 56. Description of the portions common to those in the first embodiment will be omitted.

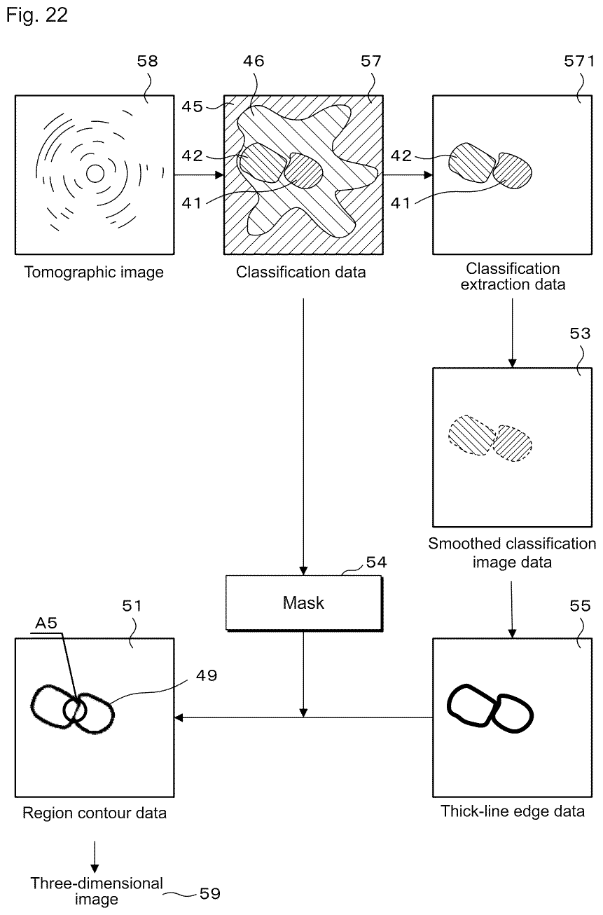

FIG. 22 is an explanatory diagram describing an outline of a process for processing a tomographic image 58 according to the sixth embodiment. The control unit 201 generates classification data 57 based on the tomographic image 58. The control unit 201 generates classification extraction data 571 from the classification data 57.

The control unit 201 applies a known smoothing filter to a classification extraction image generated based on the classification extraction data 571 to generate a smoothed classification image data 53. The smoothed classification image data 53 is image data corresponding to a smoothed classification image in which a vicinity of a boundary line of the classification extraction image is changed to gradation between a first color or a second color and a background color. In the following description, a case where the background color is white will be described as an example. In FIG. 22, a boundary line blurred due to the gradation is schematically indicated by a dotted line.

As the smoothing filter, for example, a Gaussian blur filter, an averaging filter, a median filter, or the like can be used. Since the smoothing filter is generally used in image processing, a detailed description of the smoothing filter will be omitted.

The control unit 201 generates a thick-line edge data 55 by applying a known edge extraction filter to the smoothed classification image data 53. The thick-line edge data 55 of the present embodiment can be, for example, image data displayed on a white background, with a boundary line in the classification data 57 being blurred to be a thick line.

The control unit 201 generates a mask 54 based on the tomographic image 58 and the classification data 57. The control unit 201 applies the mask 54 to the thick-line edge data 55 to generate region contour data 51. The control unit 201 generates a three-dimensional image 59 based on the region contour data 51 generated based on each tomographic image 58.

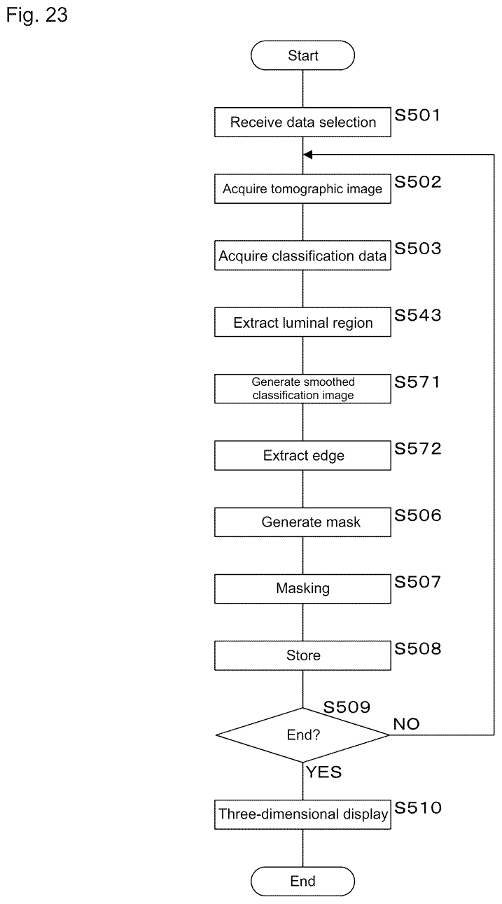

FIG. 23 is a flowchart describing a flow of processing of a program according to the sixth embodiment. Since the processing from step S501 to step S543 is similar to the processing in the first embodiment described with reference to FIG. 5, the description of the processing from step S501 to step S543 will be omitted.

The control unit 201 generates a classification image based on the classification data 57. The control unit 201 applies the smoothing filter to the classification image to generate the smoothed classification image data 53 (step S571). The control unit 201 applies the edge filter to the smoothed classification image data 53 to generate the thick-line edge data 55 (step S572).

The control unit 201 generates the mask 54 based on the classification data 57 (step S506). Since subsequent processing is similar to the processing in the first embodiment described with reference to FIG. 5, the description of the subsequent processing will be omitted.

In the first embodiment, comparatively great complexity is required for the processing for generating the thick-line edge data 55 based on the edge data 56. According to the present embodiment, the complexity of the processing for generating the thick-line edge data 55 can be significantly reduced as compared with the first embodiment. Therefore, it is possible to provide the information processing apparatus 200 that generates and displays the three-dimensional image 59 at high speed.

### Modification

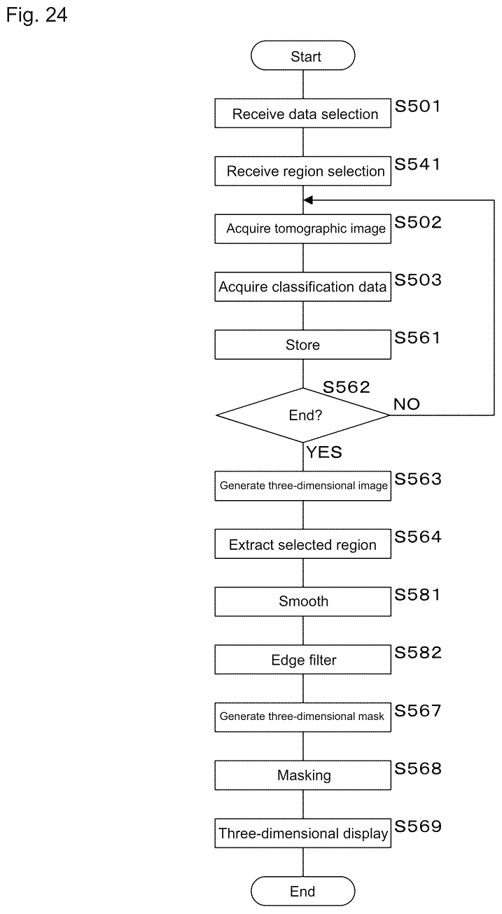

In the present modification, smoothing processing is performed on a region three-dimensional image, and thick boundary surface data is generated. FIG. 24 is a flowchart describing a flow of processing of the program according to the modification. Since the flow of the processing up to step S564 is identical to the processing of the program in the fifth embodiment described with reference to FIG. 18, the description of the flow of the processing up to step S564 will be omitted.

The control unit 201 generates a smoothed three-dimensional image by applying the known three-dimensional smoothing filter to the region three-dimensional image (step S581). The smoothed three-dimensional image is a three-dimensional image in which the vicinity of the boundary surface of the region three-dimensional image is changed to gradation between the first color or the second color and the background color. In the following description, a case where the background color is white will be described as an example.

The control unit 201 generates thick boundary surface data by applying a known three-dimensional edge filter to the region three-dimensional image (step S582). The thick boundary surface data is a three-dimensional image with a thick membrane that indicates the boundary surface between the luminal region 48 and the living tissue region 46 and is disposed in a three-dimensional space. Since the three-dimensional edge filter is known, a detailed description of the three-dimensional edge filter will be omitted.

The control unit 201 generates the three-dimensional mask 54 based on the three-dimensional image 59 generated in step S563 (step S567). Since subsequent processing is similar to the processing in the fifth embodiment described with reference to FIG. 18, the description of the subsequent processing will be omitted.

### Seventh Embodiment

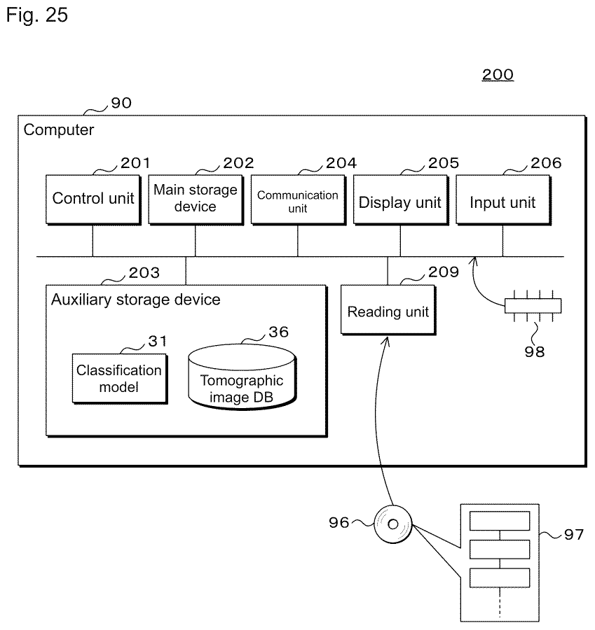

FIG. 25 is an explanatory diagram describing a configuration of an information processing apparatus 200 according to a seventh embodiment. The present embodiment relates to a mode for implementing the information processing apparatus 200 of the present embodiment by operating a general-purpose computer 90 and a program 97 in combination. Description of the portions common to those in the first embodiment will be omitted.

The computer 90 includes a reading unit 209 in addition to the control unit 201, the main storage device 202, the auxiliary storage device 203, the communication unit 204, the display unit 205, the input unit 206, and the bus described above.

The program 97 is recorded in a portable recording medium 96. The control unit 201 reads the program 97 via the reading unit 209 and stores the program in the auxiliary storage device 203. Further, the control unit 201 may read the program 97 stored in a semiconductor memory 98 such as a flash memory mounted in the computer 90. In addition, the control unit 201 may download the program 97 from another server computer, not illustrated, connected via the communication unit 204 and a network, and store the program in the auxiliary storage device 203.

The program 97 is installed as a control program of the computer 90, and is loaded in the main storage device 202 and executed. As described above, the information processing apparatus 200 described in the first embodiment is implemented. The program 97 of the present embodiment is an example of a program product.

### Eighth Embodiment

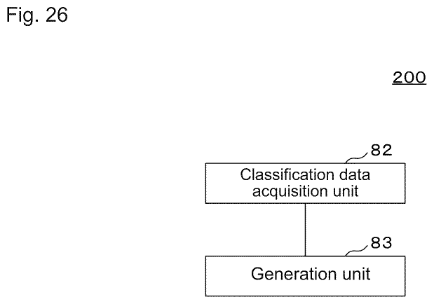

FIG. 26 is a functional block diagram of an information processing apparatus 200 according to an eighth embodiment. The information processing apparatus 200 includes a classification data acquisition unit 82 and a generation unit 83.

The classification data acquisition unit 82 acquires classification data 57 in which pixels constituting biomedical image data 58 indicating the internal structure of a living body are classified into a plurality of regions including a living tissue region 46 where a luminal region 48 exists, the luminal region 48, and an extraluminal region 45 outside the living tissue region 46. Based on the classification data 57, a generation unit 83 generates region contour data 51 obtained by removing a portion having a thickness exceeding a predetermined threshold in the living tissue region 46.

The technical features (components) described in the embodiments can be combined with each other, and new technical features can be formed by the combination.

It should be considered that the embodiments disclosed herein are examples in all respects and are not restrictive. The scope of the present invention is defined not by the meanings described above but by the claims, and is intended to include meanings equivalent to the claims and all modifications within the scope.

The detailed description above describes embodiments of an information processing method, an information processing apparatus, and a program. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. An information processing method for causing a computer to execute processing, the processing comprising:
acquiring classification data in which pixels constituting biomedical image data indicating an internal structure of a living body are classified into a plurality of regions, the plurality of regions including a living tissue region in which a luminal region exists, the luminal region, and an extraluminal region outside the living tissue region; and
generating region contour data in which a portion where a thickness from an inner surface of the living tissue region facing the luminal region exceeds a predetermined threshold is removed from the living tissue region, based on the classification data.

2. The information processing method according to claim 1, further comprising:
generating, based on the classification data, thick-line edge data in which a boundary between the living tissue region and the luminal region has a thickness within a predetermined range;
generating, based on the classification data, a mask corresponding to a pixel group classified as the living tissue region in the biomedical image data; and
applying the mask to the thick-line edge data and the region contour data.

3. The information processing method according to claim 1, further comprising:
acquiring, as the classification data, three-dimensional classification data for the three-dimensional biomedical image data;
generating, based on the three-dimensional classification data, thick boundary surface data in which a boundary surface between the living tissue region and the luminal region has a thickness within a predetermined range;
generating, based on the three-dimensional classification data, a three-dimensional mask corresponding to a pixel group classified as the living tissue region in the biomedical image data; and
applying the mask to the thick boundary surface data and generating the three-dimensional region contour data as the region contour data.

4. The information processing method according to claim 3, further comprising:
generating the thick boundary surface data by giving a thickness within the predetermined range to three-dimensional edge data generated by applying an edge extraction filter to classification image data generated based on the three-dimensional classification data.

5. The information processing method according to claim 4, further comprising:
generating the thick boundary surface data by applying a three-dimensional expansion filter to the edge data.

6. The information processing method according to claim 3, further comprising:
generating the thick boundary surface data by:
applying a three-dimensional smoothing filter to classification image data generated based on the three-dimensional classification data and generating three-dimensional smoothed classification image data; and
applying a three-dimensional edge extraction filter to the smoothed classification image data.

7. The information processing method according to claim 2, wherein the mask makes the pixel group classified as the living tissue region in the biomedical image data transparent and makes a pixel group classified as a non-living tissue region opaque.

8. The information processing method according to claim 1, wherein the classification data is data in which pixels constituting tomographic image data of a living body acquired utilizing a medical image diagnostic apparatus are classified into the plurality of regions so as to structure the biomedical image data.

9. The information processing method according to claim 1, wherein
the biomedical image data is structured from tomographic image data of a living body acquired utilizing an image acquisition catheter; and
wherein the classification data is data in which the luminal region is further classified into a first luminal region into which the image acquisition catheter is inserted and a second luminal region into which the image acquisition catheter is not inserted.

10. The information processing method according to claim 9, further comprising:
receiving from the first luminal region and the second luminal region, selection of one or more selected regions; and
generating thick-line edge data having a thickness within a predetermined range at the boundary between the living tissue region and the luminal region, or thick boundary surface data having a thickness within a predetermined range based on the three-dimensional classification data for the three-dimensional biomedical image data acquired as the classification data, based on a boundary or a boundary surface between the living tissue region and the selected region.

11. The information processing method according to claim 1, wherein
the biomedical image data includes a plurality of pieces of the tomographic image data generated in time series;
wherein the classified data includes a plurality of pieces of two-dimensional classification data generated respectively based on the plurality of pieces of the tomographic image data;
wherein the region contour data includes a plurality of pieces of two-dimensional region contour data generated respectively based on the plurality of pieces of two-dimensional classification data; and
generating based on the two-dimensional region contour data, a three-dimensional image.

12. The information processing method according to claim 1, wherein
the biomedical image data is three-dimensional biomedical image data structured from a plurality of pieces of the tomographic image data generated in time series;
the classification data includes three-dimensional classification data for the three-dimensional biomedical image data;
the region contour data includes three-dimensional region contour data generated based on the three-dimensional classification data; and
generating a three-dimensional image based on the three-dimensional region contour data.

13. The information processing method according to claim 11, further comprising:
acquiring, based on the classification data, thickness information about the living tissue region;
wherein in the region contour data, giving display color data corresponding to the thickness information to pixels corresponding to at least an in-contour surface or an outer surface; and
generating the three-dimensional image based on the region contour data and the display color data.

14. An information processing apparatus, comprising:
a classification data acquisition unit configured to acquire classification data in which pixels constituting biomedical image data indicating an internal structure of a living body are classified into a plurality of regions including a living tissue region in which a luminal region exists, the luminal region, and an extraluminal region outside the living tissue region; and
a generation unit configured to generate region contour data in which a portion where a thickness exceeds a predetermined threshold is removed from the living tissue region, based on the classification data.

15. The information processing apparatus according to claim 14, further comprising:
an output unit configured to output a three-dimensional image generated based on the region contour data.

16. A non-transitory computer-readable medium storing a computer program that causes a computer to execute processing, the processing comprising:
acquiring classification data in which pixels constituting biomedical image data indicating an internal structure of a living body are classified into a plurality of regions including a living tissue region in which a luminal region exists, the luminal region, and an extraluminal region outside the living tissue region; and
generating region contour data in which a portion where a thickness exceeds a predetermined threshold is removed from the living tissue region, based on the classification data.

17. The non-transitory computer-readable medium according to claim 16, further comprising:
generating, based on the classification data, thick-line edge data in which a boundary between the living tissue region and the luminal region has a thickness within a predetermined range;
generating, based on the classification data, a mask corresponding to a pixel group classified as the living tissue region in the biomedical image data; and
applying the mask to the thick-line edge data and the region contour data.

18. The non-transitory computer-readable medium according to claim 16, further comprising:
acquiring, as the classification data, three-dimensional classification data for the three-dimensional biomedical image data;
generating, based on the three-dimensional classification data, thick boundary surface data in which a boundary surface between the living tissue region and the luminal region has a thickness within a predetermined range;
generating, based on the three-dimensional classification data, a three-dimensional mask corresponding to a pixel group classified as the living tissue region in the biomedical image data; and
applying the mask to the thick boundary surface data and generating the three-dimensional region contour data as the region contour data.

19. The non-transitory computer-readable medium according to claim 18, further comprising:
generating the thick boundary surface data by giving a thickness within the predetermined range to three-dimensional edge data generated by applying an edge extraction filter to classification image data generated based on the three-dimensional classification data.

20. The non-transitory computer-readable medium according to claim 19, further comprising:
generating the thick boundary surface data by applying a three-dimensional expansion filter to the edge data.
